(19) United States
(12) Patent Application Publication
(10) Pub. No.: US 2022/0076454 A1
(43) Pub. Date: Mar. 10, 2022

(54) HYPERSPECTRAL TESTING

(71) Applicant: Radient Technologies Innovations Inc., Edmonton (CA)

(72) Inventors: Michael Cabigon, Edmonton (CA); Jim Seethram, Edmonton (CA); Steven Splinter, North Vancouver (CA); Denis Taschuk, Edmonton (CA)

(21) Appl. No.: 17/238,068

(22) Filed: Apr. 22, 2021

Related U.S. Application Data
(63) Continuation of application No. PCT/IB2019/058783, filed on Oct. 15, 2019.
(60) Provisional application No. 62/749,032, filed on Oct. 22, 2018.

(51) Int. Cl. G06T 7/00; G01N 33/00; G01N 21/25; G06Q 50/02; G06Q 10/04

(52) U.S. Cl. CPC G06T 7/97; G01N 33/0098; G06T 2207/10036; G06Q 50/02; G06Q 10/04; G01N 21/255

(57) ABSTRACT

Methods and systems for determining a recommended harvest date of a Cannabis biomass are disclosed. The methods and systems include a research network having a plurality of databases and a plurality of software modules stored thereon; a grower base module operable to send data to, and receive data from, the research network via a communication network; and an extractor base module operable to evaluate an extract and send data to the research network via the communication network. An optical sensor, including a hyperspectral or multispectral camera, provides current images of the Cannabis crop. An analyzer module and time-to-harvest module may be used to determine a recommended harvest date based on historical data.

### CROSS-REFERENCE TO RELATED APPLICATIONS

The present patent application is a continuation of PCT/IB2019/058783 filed Oct. 15, 2019 which claims the priority benefit of U.S. provisional patent application No. 62/749,032 filed Oct. 22, 2018, the disclosures of which are incorporated by reference herein.

### BACKGROUND OF THE INVENTION

### 1. Field of the Disclosure

The present disclosure is generally related to plant analytics. More specifically, the present disclosure relates to a method for improving the yield of desirable elements of a Cannabis plant by analyzing growth pattern indicators.

### 2. Description of Related Art

Cannabis is a genus belonging to the family cannabaceae. There are three common species of Cannabis including Cannabis sativa, Cannabis indica, and Cannabis ruderalis. The genus cannabaceae is indigenous to Central Asia and the Indian subcontinent and has a long history of being used for medicinal, therapeutic, and recreational purposes. For example, Cannabis is known to be capable of relieving nausea (such as that accompanying chemotherapy), pain, vomiting, spasticity in multiple sclerosis, and increase hunger in anorexia. The term Cannabis as used herein can refer to a “Cannabis biomass” which can encompass the Cannabis sativa plant and variants thereof, including subspecies sativa, indica and ruderalis, Cannabis cultivars, and Cannabis chemovars (varieties characterised by chemical composition). The term “Cannabis biomass” is to be interpreted accordingly as encompassing plant material derived from one or more Cannabis plants. Such Cannabis biomasses can naturally contain different amounts of the individual cannabinoids.

Each Cannabis biomass contains a unique class of terpeno-phenolic compounds known as cannabinoids, or phytocannabinoids. The principle cannabinoids present in a Cannabis biomass can include Delta-9-tetrahydrocannabinolic acid (THCA) and cannabidiolic acid (CBDA). THCA does not include psychoactive properties on it's own, but when decarboxylated THCA becomes Delta-9-tetrahydrocannabinol (THC), which is a potent psychoactive cannabinoid. CBDA can be decarboxylated into cannabidiol (CBD), which is a major cannabinoid substituent in hemp Cannabis. CBD is a non-psychoactive cannabinoid and is widely known to have therapeutic potential for a variety of medical conditions including, but not limited to, those described above.

Historical delivery methods of cannabinoids have included combustion (such as smoking) of the dried Cannabis plant material, or biomass. However, smoking can result in adverse effects on a user's respiratory system due to the production of potentially toxic substances. Moreover, smoking is an inefficient mechanism which delivers a variable mixture of both active and inactive substances, many of which may be undesirable. Common alternative delivery methods, including but not limited to, ingestion, typically require an extraction process to be performed on the Cannabis biomass to remove the desired components. Such ingestible Cannabis items can include, but are not limited to, concentrates, extractants, and Cannabis oils. Often, such ingestible Cannabis items, or Cannabis extracts, are formulated using convenient pharmacologically acceptable diluents, carriers, and/or excipients to produce a composition, which are collectively known as Cannabis derivative products.

Cannabis extracts, as described above, can be obtained from a Cannabis biomass by any number of methods including, but not limited to, supercritical fluid extraction, solvent extraction, and microwave-assisted extraction. In most cases, the yield of Cannabis extract obtained, and thus the yield of the extracted product, depends upon the cannabinoid composition of the original Cannabis biomass.

A chemical analysis of the whole, or a portion of, Cannabis plants can be used to determine the composition of the Cannabis biomass, including the concentration of cannabinoids and other active components. However, these methods are typically destructive in nature, requiring that the plants be processed and then analyzed by methods such as High Performance Liquid Chromatography (HPLC) or other similar analytical techniques. As such, a non-destructive method for assessing the quality of the plant material is needed to allow for in planta or in natura determination of biomass quality.

### SUMMARY OF THE CLAIMED INVENTION

Examples of the present disclosure provide systems and methods for determining a recommended harvest date for Cannabis biomass. In particular, a system for evaluating the recommended harvest date can include a grower base module, an extractor base module, and a research network communicable coupled with one another via a communication network. The research network can have historical data relating to previous Cannabis crops stored thereon, the historical data can include optical images of previous crops as well as information regarding the final extracted product. The use of the historical data can provide an increase in the volume and quality of the final product extracted from the Cannabis biomass.

In addition to improving the quality of the cannabinoid extract, such systems and methods can further provide growers with information regarding ideal conditions for growing their crops and recommended harvest timing. The systems can further allow growers and extractors to communicate with one another throughout the growth cycle of the crop in order to improve the quality of the Cannabis crop and the resulting extract.

### BRIEF DESCRIPTIONS OF THE DRAWINGS

FIG. 1 illustrates an exemplary network environment in which a Cannabis growth monitoring system may be implemented.

FIG. 2 is a diagrammatic view illustrating an exemplary hyperspectral image analyses.

FIG. 3 is a flowchart illustrating an exemplary method for creating a Cannabis volume-quality database in accordance with the present disclosure;

FIG. 4 is a flowchart illustrating an exemplary method for providing a recommended harvest date;

FIG. 5 is a flowchart illustrating an exemplary method for evaluating the volume and quality of cannabinoids in accordance with the present disclosure;

FIG. 6 is a flowchart illustrating an exemplary method for obtaining a recommended harvest date in accordance with the present disclosure; and

FIG. 7 is a flow chart illustrating an exemplary method determining the volume and quality of cannabinoids in a crop in accordance with the present disclosure.

### DETAILED DESCRIPTION

The volume and quality of the cannabinoids present in an individual plant or biomass can vary from cultivar to cultivar, as well as from season to season. Not only can the volume and quality vary, but the concentration of cannabinoids including, but not limited to, delta-9-tetrahydrocannabinolic acid (THCA) and cannabidiolic acid (CBDA), can also fluctuate. This can occur not only from cultivar to cultivar, but the proportion of cannabinoids in a single plant can vary from season to season based upon soil, climate, growing conditions and harvesting time and methods. Thus, based on the proportion of the different cannabinoids present in a particular biomass, the psychoactive and medicinal effects obtained from said plant can vary as well. Such variance can be further exacerbated by the presence of other elements, including, but not limited to, terpenoid and phenolic compounds that can also be present in the Cannabis biomass. These terpenoid and phenolic compounds can have additional pharmacological properties and benefits.

Embodiments of the present invention may include a method for assisting Cannabis growers to understand the best time to harvest the Cannabis biomass. Specifically, the method may use historical data gathered by both Cannabis growers, cannabinoid extractors, and other personnel at various processing points to determine ideal growth periods for different cultivars so as to achieve a certain type of result (e.g., preferred types, levels, or concentrations of one or more cannabinoids, terpenoids, flavonoids, or other component).

Present harvest times can be determined by using magnification devices (e.g., a loupe or microscope) to determine the amount of trichomes on Cannabis buds, or flowers. Sometimes referred to as “crystals,” trichomes are glands filled with resins made up of cannabinoids, including but not limited to THCA and CBDA, as well as flavonoids and terpenes. Trichomes can resemble tiny clear glass mushrooms, often with a stalk and a bulbous head. As the time for harvest of the Cannabis plant approaches, the trichome heads may turn from clear to a cloudy or milky white. As the Cannabis plant matures beyond ripeness, the trichomes may turn amber in color. If the Cannabis plant is harvested when the majority of the trichome heads are clear or cloudy, the compounds extractable from the resulting biomass, such as cannabinoids, may be increased. Conversely, if the grower waits until the trichomes are primarily amber, the amount and/or quality of cannabinoids (and therefore the effects of any Cannabis extraction) may be decreased. It should be noted that while the ideal harvest time are described above in relation to cloudy or milk white appearance of the trichomes, some growers prefer to harvest their plants prior to the appearance of the milky coloration, such that extractors are able to obtain a lighter-colored product that can be more desirable to some buyers in the marketplace. Additionally, because the terpenes may begin to degrade as the plant continues to mature, some producers may prefer to risk a reduction in potency by harvesting early in exchange for preserving the flavor and clarity of their extracts.

The present disclosure provides a method for increased accuracy in estimating trichome maturation. Such estimated maturation may further be used to determine when a grower should harvest the Cannabis plant, as well as predict what quality level and yield to expect from the crop based on time of harvest. Currently, growers do not have access to a large amount of technology to assist in the determination of when to harvest the biomass. However, as the availability and variety of Cannabis plants continues to grow, increased efficiency tailored to specific desired outcomes (e.g., specific compounds, as well as concentrations and effects thereof) during harvest may be desired.

The amount of cannabinoids in the extracted product may be directly related to the amount of cannabinoids present in the Cannabis biomass at the time of harvest. As described above, current methods for testing cannabinoid content during the plant's growth cycle has generally required destruction of all or a portion of the sample. As such, a method for more accurately measuring trichome maturation without the destruction of the Cannabis biomass is needed.

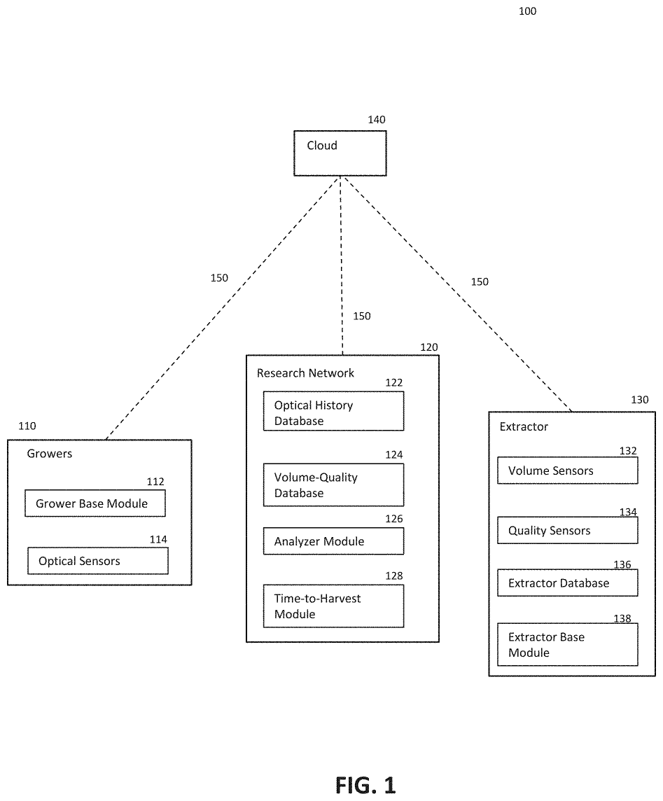

Exemplary methods and systems for determining Cannabis maturation and harvest quality are described herein. FIG. 1 illustrates an exemplary network environment in which a system 100 may be implemented for Cannabis growers devices 110 and cannabinoid extractors devices 130 to monitor, evaluate, and harvest crops in the most effective manner. The system 100 described herein can include one or more Cannabis grower devices 110, a research network server 120, and one or more Cannabis extractor devices 130 in communication with one another via connections 150 to one or more communication networks 140. The communication network 140 may be inclusive of wired and/or wireless networks. The communication network 140 may be implemented, for example, using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network 140 can allow ubiquitous access to shared pools of configurable system resources and higher-level services that can be rapidly provisioned with minimal management effort, often over Internet and relies on sharing of resources to achieve coherence and economies of scale, like a public utility, while third-party clouds may enable organizations to focus on their core businesses instead of expending resources on computer infrastructure and maintenance. In at least one example, the growers devices 110 and extractors devices 130 can access the grower base module 112, the extractor base module 138, and the research network server 120 via an application. In at least one example, the application can include an API. The API (or application programming interface) is an application-specific interface that can allow the growers devices and extractors devices to communicate with the research network server 120. The modules, databases, and networks described with respect to FIG. 1 can be stored on, and accessible via, the cloud network 140.

Growers devices 110 can include, but are not limited to, devices associated with industrial and individual Cannabis farmers, who are responsible for planting and maintaining the Cannabis plants during the maturation of the crop. For example, the growers device 110 can direct management of the growth of healthy Cannabis plants by specifying that the plants be provided with the appropriate amounts of light, nutrients, water, and other resources. The growers may have been primarily responsible for determining when to harvest the Cannabis biomass from the plants based on local conditions, as well as preparing the harvested biomass for transportation to the extractors. Whereas past systems may have relied upon subjective perspectives and opinions to determine readiness to harvest, the present system seeks to make objective measurements and apply rule-based analytics to identify harvest times that are tailored to specific goals.

The growers devices 110 can have a grower base module 112 which can be a software module operable to receive and compare optical images of the Cannabis crop throughout the growth cycle of the plants. Specifically, the growers devices 110 can take optical images of the Cannabis crop using one or more optical sensors 114 and upload the optical images to the research network server 120. It is understood that for the purposes of this disclosure, “hyperspectral analysis” or “hyperspectral testing” may be substituted for any destructive method of in-planta determination of Cannabis biomass quality, including but not limited to, image analysis, electronic noses, and/or other type of non-destructive sensors or tests. An analysis of sensor data can then be performed using an analyzer module 126 and provide growers devices 110 with analytics results, including but not limited to recommendations for harvest time.

The research network server 120 can be a server comprising an optical history database 122, a volume-quality database 124, as well as software including an analyzer module 126 and a time-to-harvest module 128. The research network can be accessible to growers devices 110 and extractors devices 130 via the cloud 140, allowing both growers devices 110 and extractors devices 130 to view and communicate with the optical history database 122 and the volume-quality database 124. An analyzer module 126 stored on the research network server 120 can include a software program operable to evaluate the optical images provided by growers devices 110 to determine on intensity vs. wavelength of a particular image and create a rating for the optical images. For example, the ratings can describe how close the optical image of the current crop is to the volume-quality database images, or ideal images. Such ratings can indicate a high, medium, or low trichome level. A research optical database is a database accessible via the cloud which is capable of storing historical records of Cannabis plant images such that the analyzer module can compare previous, or historical, images indicating quality and volume of cannabinoids.

A time-to-harvest module 128 can be stored on the research network server 120 and can be a software program operable to evaluate data provided by growers 100 to predict an optimum time for a grower 110 to harvest their Cannabis biomass. Once executed, the time-to-harvest module 128 can provide a harvest date recommendation to the growers devices 110. In at least one example, the API can allow growers devices 110 to access data including research relating to the optical history database 122 and the volume-quality database 124, see recommendations relating to harvest date, and to communicate with extractors devices 130. The images of crops sent to the research network server 120 for detailed analysis can be stored on the optical history database 122. The volume-quality database 124 is a database operable to store, by lot number, the cannabinoid concentration of a harvested Cannabis crop for throughout the growth periods as well as the analyzer module 126 results in terms of quality and volume related to cannabinoid content. The research network server 120 can later receive extraction and store such data in the volume-quality database 124. Different cannabinoid volume-quality levels can be ideal for different final Cannabis derivative products. As such, in at least one example, the time-to-harvest module 128 can take the Cannabis derivative product to be created with the extracted cannabinoids into account when providing the growers devices 110 with a recommended harvest date.

Extractors devices 130 can include any group operating a means for producing an extract or concentrate product from a harvested Cannabis biomass. Said extractors devices 130 can employ various known technologies or methods for producing cannabinoid extracts and the derivative products therefrom. The extractors devices 130 can use volume sensors 132 and quality sensors 134 to determine the volume and quality of the final extract obtained from the Cannabis biomass. An extractor base module 138 can be used to determine the effectiveness of grower's process by evaluating the optical images of the Cannabis plants, executing the analyzer module 126, executing the time-to-harvest module 136 and outputting the results obtained relating to cannabinoid levels and suggested harvest date. The extractor base module 138 can also allow extractors devices 130 to communicate with growers 130 via the communication network 140.

During the extraction process, extractors remove and concentrate the terpeno-phenolic compounds from the harvested Cannabis biomass. As described above, the terpeno-phenolic compounds can include, but are not limited to, cannabinoids such as THCA and CBDA. The amount of product extracted from the Cannabis biomass can be quantified in terms of a yield. The “yield” produced by the extractors refers to the amount, or mass, of product extracted from the Cannabis biomass, as compared to the total mass of Cannabis biomass. For example, a 100% yield would imply that the mass of the extracted product equaled the mass of the initial biomass. A yield of 100% is highly unlikely, a more realistic scenario is obtaining 90 grams (g) of extract from 454 g (1 pound) of biomass. Such extraction would equate to a “yield” of approximately 20% (90 g divided by 454 g=19.8%). However, yield of the Cannabis biomass is practically meaningless without relating said yield to the quality or potency of the extracted product.

The extracted cannabinoids can be used in the production of various Cannabis products which often are marketed and sold based upon the specific cannabinoid potency or cannabinoid profile. The most common cannabinoid content identified in backing includes, but is no limited to delta-9-tetrahydrocannabinol (THC), cannabidiol (CBD), and cannabinol (CBN). However, the extraction “yield” as described above is not equivalent to a measurement of the specific cannabinoid content. For example, the extraction product yield does not provide any insight as to the THC content of the extraction product. As such, the potency, the amount or concentration of a specific cannabinoid, is the most critical measurement in the marketing of a Cannabis product. The amount of cannabinoids in the extracted product can be directly related to the amount of cannabinoids present in the harvested Cannabis biomass and the effectiveness, or efficiency, of the extraction process. In at least one example, a Cannabis biomass can have THC potencies that range from about 0% to as high as about 30%. Assuming the extraction process can achieve the removal of all, or nearly all, of the THC in the Cannabis biomass, then higher the THC content of the starting Cannabis biomass, the higher the THC content of the extracted product and the higher the yield of extracted product.

While the description of FIG. 1 general refers to Cannabis as grown by “growers” and Cannabis extracts and derivative products as produced by “extractors,” it should be recognized that in some cases, the grower and the extractor may be the same entity.

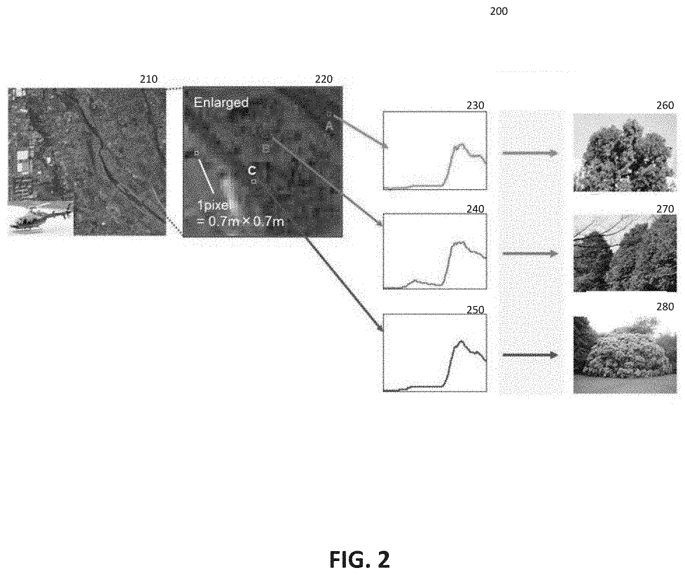

An exemplary system for analyzing the volume and quality of a Cannabis crop can include a plurality of optical sensors, as described in FIG. 1, placed throughout a Cannabis crop and used to take optical images of the Cannabis plants throughout the plant growth cycle. In an alternative example, a drone or other mobile sensor can be used to retrieve optical images at predetermined periods during the growth cycle of the crops. The optical images can then be analyzed to determine the volume and quality of the cannabinoids of the Cannabis plant. For example, FIG. 2 illustrates a system 200 for analyzing a Cannabis crop beginning with obtaining an optical image 210 of at least a portion of the Cannabis crop. The optical image 210 is merely used as exemplary for the purposes of description and is not to be considered as limiting. In at least one example, hyperspectral and multispectral imaging can be used to obtain the optical images. As used herein, the term “hyperspectral imaging” refers to spectral imaging which collects and processes information from across the electromagnetic spectrum. As used herein, the term “multispectral imaging” refers to a type of imaging which captures data within a specific wavelength range across the electromagnetic spectrum. In at least one example, multispectral images can be obtained which are then analyzed using hyperspectral analysis techniques. For example, a multispectral imaging device can be tuned to specific frequencies depending on what compounds the grower wishes to analyze.

Specifically, hyperspectral imaging, like other spectral imaging, collects and processes information from across the electromagnetic spectrum. The goal of hyperspectral imaging is to obtain the spectrum for each pixel in the image of a scene, with the purpose of finding objects, identifying materials, or detecting processes. There are two general branches of spectral imagers, push broom scanners and the related whisk broom scanners, which read images over time, and snapshot hyperspectral imaging, which uses a staring array to generate an image in an instant.

Human eye sees color of visible light in mostly three bands (long wavelengths—perceived as red, medium wavelengths—perceived as green, and short wavelengths—perceived as blue), spectral imaging is capable of dividing the spectrum into many more bands. This technique of dividing images into bands can be extended beyond the visible. In hyperspectral imaging, the recorded spectra have fine wavelength resolution and cover a wide range of wavelengths. Hyperspectral imaging measures contiguous spectral bands, as opposed to multispectral imaging which measures spaced spectral bands. Engineers can build hyperspectral sensors and processing systems for applications in astronomy, agriculture, molecular biology, biomedical imaging, geosciences, physics, and surveillance. Such hyperspectral sensors look at objects using a vast portion of the electromagnetic spectrum. Certain objects can leave unique ‘fingerprints’ in the electromagnetic spectrum. Known as spectral signatures, these ‘fingerprints’ enable identification of the materials that make up a scanned object.

For Cannabis analysis, specific electromagnetic frequencies have been determined to be indicative of different cannabinoid quality when analyzed using hyperspectral analysis (for example, the percentage of THC present on the flowers of the Cannabis plant). For example, THC can produce a unique fingerprint under the electromagnetic spectrum. As such, if a grower wants to evaluate the volume or quality of THC in their crop, the grower can set the optical sensors to the spectrum relating to THC for analysis. It is to be understood that for the purposes of this disclosure, THC is considered exemplary and may be substituted by any of the other cannabinoids present in Cannabis plants, including the acid forms, which may be of interest for hyperspectral testing.

Referring back to FIG. 2, an analysis of the optical image 210 of the Cannabis crop can begin by enlarging 200 various portions of the image such that the individual pixels of the optical image 210 can be analyzed. In order to properly evaluate the volume and quality of the Cannabis crop, the optical image 210 can be embedded with location data such that the cannabinoid analysis can be recorded as corresponding with a particular section of the crop. The enlarged image 220 can be analyzed using the electromagnetic spectrum to determine the volume and quality of plants. For example, the analysis can determine whether the crops in a specific location include plants corresponding to various electromagnetic ranges, as shown in images 230, 240, 250.

The graphs provided as images 230, 240, 250 illustrate wavelength versus intensity detected by the hyperspectral camera. Each image may correspond to a different part of the Cannabis plant growth cycle. Each stage may exhibit different variations in color intensity for each wavelength examined. Cannabis plants (for a unit volume=10 ft by 10 ft) may exhibit different characteristics as detected in different optical images, as more and more buds appear and as the color of the buds appear and change. In this way, such images can be used later to correlate wavelength-intensity to lab analyses indicative of specific extraction results, including types of compounds that are extracted, as well as concentrations and properties thereof. Over time, a database of images may be accumulated and used as references for comparison to later-captured images, and such comparisons may be used to make predictions as to the resulting extracts.

It may be necessary to normalize the total intensity level (high, medium, low) to a standard (not shown) to allow better understanding of the graphs. The electromagnetic ranges can then be embedded in the optical image, and the optical image can be loaded to an optical history database for storage. As described above, the optical history database can be a database operable to store historical optical image data of Cannabis crops by lot number. The optical database can include the optical images of the crops taken during the plant growth cycle, as well as related data. Such data can include extraction data including, but not limited to, volume and quality of the final extracted product. The volume and quality data can be obtained from the extractors, correlated to the optical images 210, and saved in a volume-quality database as described with respect to FIG. 1.

A table showing an exemplary entry of the optical history database is provided as Table 1, below.

TABLE 1

| Lot | Days since | Optical Image Data Files | | | | Extractor Data | |
|---|---|---|---|---|---|---|---|
| Number | germination | Day 1 | Day 2 | Day 3 | Day n | Quality | Volume |
| 71 | 38 | L71D1 | L71D2 | L71D3 | L71Dn | High | High |
| 121 | 32 | L121D1 | L121D2 | L121D3 | L121Dn | High | Medium |
| . . . | . . . | . . . | . . . | . . . | . . . | . . . | . . . |
| . . . | . . . | . . . | . . . | . . . | . . . | . . . | . . . |
| 201 | 38 | L201D1 | L201D2 | L201D3 | L201Dn | Medium | Low |

The data provided with respect to Table 1 is merely exemplary and is not to be considered as limiting. As shown, the optical history database can relate the lot number of the Cannabis biomass to the growth cycle of the plant based on germination date. Depending on the number of optical images provided by the growers, such optical images can be uploaded daily to be used in the analysis. The optical history database can additionally be updated once the extraction is complete and actual cannabinoid volume and quality may be determined as described in detail below. Various thresholds may be set for the qualitative descriptors, which may be tailored to the specific cultivar and/or extracted compound. Background data regarding growing conditions upstream and specific types of downstream data—extracted compounds, concentrations, quality levels, effects thereof, etc.—may be tracked and added to the database for more granular analysis as to what conditions and factors are more or less likely to lead to such extraction results.

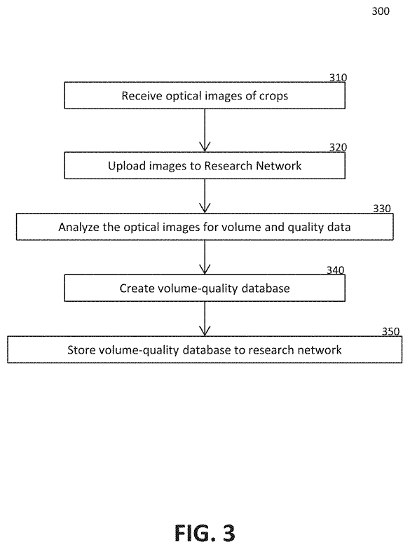

An exemplary method 300 for creating the volume-quality database is illustrated in FIG. 3. One skilled in the art will appreciate that, for this and other processes and methods disclosed herein, the functions performed may be implemented in a different order than the one described herein. The outlined steps and operations are only provided as examples, and some of the steps and operations may be optional, combined into fewer steps and operations, or expanded into additional steps and operations without detracting from the essence of the disclosed embodiments. The method 300 can begin at block 310 where optical images of a crop are received. For example, optical images taken by growers throughout the Cannabis growth cycle, as described with respect to FIG. 2. In at least one example, optical images can be provided at periodic intervals determined by the grower. In an alternative example, the sensors can be programmed to continuously obtain optical images of the crops (such as daily, weekly, etc.). At block 320, the optical images can be uploaded to a research network, as described with respect to FIG. 1. The optical images can be embedded with plant data including, but not limited to, cultivar, germination date, and the like.

At block 330, the optical images can be analyzed for volume and quality data. When a Cannabis biomass is subjected to an extraction process, the end product can contain various elements including, but not limited to, cannabinoids, terpenes, oils, waxes, lipids, proteins, and other plant material. The total amount or mass of the extracted product has little to do with the actual value and efficacy of the product. The composition of each of the various elements described above determines the potency, effect, and value of the extract. For example, Extract Company A processed 10 kg of Cannabis biomass and obtained a yield of 20%. An analysis of the extracted product was shown to contain 20% THC. Extract Company B took the same Cannabis biomass and obtained a lower yield, but the final product contained a higher concentration of THC. Based upon an analysis of “yield” alone, Extract Company A was superior to Extract Company B. However, the lower purity of the extracted product from Extract Company A suggests the Cannabis biomass used contained more waste material, thus increasing its final mass and the resulting yield.

As such, a much more important evaluation tool is “extraction efficiency.” Extraction efficiency is the percentage of desired substance, such as CBD or THC, that is actually extracted as compared to how much of the desired substance was available in the original Cannabis biomass. One or more extractor volume sensors can be used to measure the exact weight of the desired compounds. The extractor “quality,” or “cannabinoid potency,” reflects data which quantifies the level of plant cannabinoids present in the final Cannabis derivative products. Cannabis derivative product producers are required by regulation to obtain and provide customers with potency data for the cannabinoids in their products. It's important for consumers to know cannabinoid levels, such as THC and CBD levels, since they can have a strong influence on the resulting effects of the Cannabis derivative product. For example, some medical patients may want a product with a high CBD:THC ratio, due to the medicinal value of CBD. Others may prefer the opposite, a high THC:CBD ratio due to the psychoactive effects of THC.

Various methods for measuring cannabinoid concentration are known in the art. The most common techniques for measuring cannabinoids is High Performance Liquid Chromatography (HPLC) or Ultra High Performance Liquid Chromatography (UHPLC). HPLC is capable of quantifying the amount of active compounds in the Cannabis derivative product. An extractor database 136 is a database which maintains data associated with the extraction process and resulting efficiency. Specifically, the extractor database 136 can store data by lot number of Cannabis biomass sources, extractor quality, and volume sensors data.

At block 340, a volume-quality database can be created based on the analyzed images. The volume-quality database can be used to store data by lot number, for quality and volume ratings for the Cannabis crops on a per-image basis. The quality and volume data can related to extracted elements including, but not limited to, THC, CBD, CBN, flavonoids, terpenes and an overall volume and quality of the trichomes. The analysis of the optical images can be performed by the analyzer module, described above. The analyzer module can determine the trichomes levels of a plurality of factors including, but not limited to, THC, CBD, CBN, flavonoids, and terpenes. This data can then be converted using a standard to provide quality and volume ratings, such as High, Medium, and Low. Once the standard volume and quality data is determined, the data can be related back to the lot number of the Cannabis biomass and stored in the volume-quality database. Table 2, below, illustrates an exemplary volume-quality database entry showing the standardized levels of the various elements. The data provided in Table 2 is merely provided for the purposes of this description and should not be considered limiting.

TABLE 2

| | | Quality | | | | | | Volume | | | | | |
|---|---|---|---|---|---|---|---|---|---|---|---|---|---|
| Lot | Day | THC | CBD | CBN | Flavonoids | Terpenes | Overall | THC | CBD | CBN | Flavonoids | Terpenes | Overall |
| 71 | 1 | Low | Low | Low | Low | Low | Low | Low | Low | Low | Low | Low | Low |
| 71 | 2 | Low | Low | Low | Low | Low | Low | Low | Low | Low | Low | Low | Low |
| 71 | 3 | Low | Low | Low | Low | Low | Low | Low | Low | Low | Low | Low | Low |
| 71 | 4 | Low | Low | Low | Low | Low | Low | Low | Low | Low | Low | Low | Low |
| . . . | . . . | . . . | . . . | . . . | . . . | . . . | . . . | . . . | . . . | . . . | . . . | . . . | . . . |
| . . . | . . . | . . . | . . . | . . . | . . . | . . . | . . . | . . . | . . . | . . . | . . . | . . . | . . . |
| 71 | 34 | High | High | Med | Med | High | Med | Med | Med | Med | High | Med | Med |
| 71 | 35 | High | High | High | High | High | High | Med | Med | High | High | Med | Med |
| 121 | 1 | Low | Low | Low | Low | Low | Low | Low | Low | Low | Low | Low | Low |
| 121 | 2 | Low | Low | Low | Low | Low | Low | Low | Low | Low | Low | Low | Low |
| 121 | 3 | Low | Low | Low | Low | Low | Low | Low | Low | Low | Low | Low | Low |
| 121 | 4 | Low | Low | Low | Low | Low | Low | Low | Low | Low | Low | Low | Low |
| . . . | . . . | . . . | . . . | . . . | . . . | . . . | . . . | . . . | . . . | . . . | . . . | . . . | . . . |
| . . . | . . . | . . . | . . . | . . . | . . . | . . . | . . . | . . . | . . . | . . . | . . . | . . . | . . . |
| 121 | 37 | High | High | Med | Med | High | Med | Med | Med | Med | High | Med | Med |
| 121 | 38 | High | High | High | High | High | High | High | High | High | High | Med | High |

Finally, at block 350, the volume-quality database can be stored on the research network. The volume-quality database can be accessed by both growers and extractors, as described with respect to FIG. 1. Access to the volume-quality database can provide growers with images showing ideal volume and quality conditions for harvest, as described in more detail below.

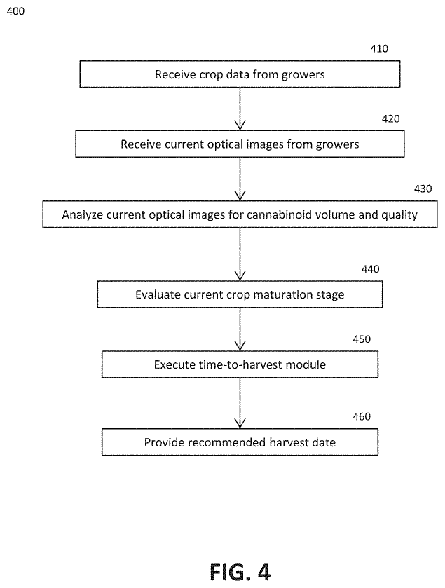

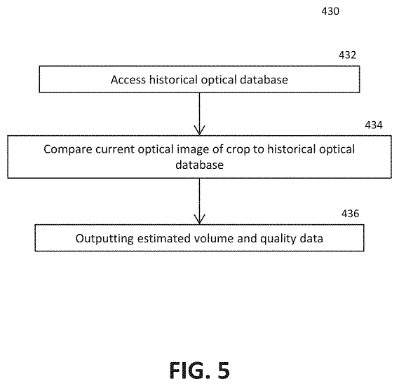

The volume-quality database can be used to assist in the optical image analysis described with respect to FIG. 2. For example, an exemplary method 400 for providing a grower with a recommended harvest date is illustrated in FIG. 4. The method 400 can begin at block 410 where crop data is received from growers. The crop data can include, but is not limited to cultivar, germination date, light levels, nutrient levels, water levels, and the like. The crop data can be uploaded and stored in the research network. At block 420 current optical images of the Cannabis crops can be received at the research network from the growers. The optical images provided can be hyperspectral or multispectral images as described in detail above. At block 430, the current optical images are analyzed for cannabinoid volume and quantity. A detailed method for the analysis of the optical images is provided with respect to FIG. 5.

Specifically, at block 432 of FIG. 5, the historical optical image database, described above, is accessed to retrieve historical optical images of crops in various states of maturation. The optical history database, as described with respect to FIG. 1, is a database which is operable to store information, by lot number, of Cannabis plants. The information can include, but is not limited to, the number of days in the plant's growth cycle and the date the crop was ultimately harvested. The harvest date can be stored as a number of days after germination. Added to this database, for each lot number is the final Cannabis volume and quantity as measured by an extractor. The optical history database can include data relating to several lot numbers and growth cycles. The optical history database can also store the optical images (frequency or wavelength and associated intensity) provided for each day in the cycle of the lot number. In this way, it is possible to take a current series of images of a current growth cycle and compare it to all the past series to match image. The historical optical images elected for comparison can be selected for various reasons including, but not limited to, cultivar, crop location, and other factors relevant to the maturation of the trichomes of the Cannabis plants.

At block 434, the current optical images are compared to the crop images from the historical optical database to determine which historical photo most accurately corresponds to the current optical image. The optical images can be compared using an analyzer module as described above. The analyzer module can access the optical history database of the research network to locate historical optical images and having trichome formation similar to the current optical image. The photos can be analyzed to determine a most similar trichome size, color, quantity, etc. The trichomes correlate to the volume and quality of the cannabinoids available for extraction and are thus useful in the present analysis. For example, during a research study, many strains, or cultivars, of Cannabis can be used multiple times in a growth cycle. For each image taken of the crop, a laboratory analysis (such as HPLC) can be performed to determine cannabinoid volume (e.g. concentration) and quality (e.g. cannabinoid profile). The volume and quality data can then be associated, or embedded, in the historical optical image. In this way, analyzer module can allow for a new image to be matched to a closest historical optical image to output the historical volume and quality data as an estimated value for the current crop. At block 436, an estimated volume and quality for the current crop is provided back into method 400 for maturation stage determination.

Referring back to FIG. 4, at block 440 the maturation stage of the crops is determined based on the volume and quality estimates provided by the analyzer module. Comparing the trichrome stage with photos from the optical history database can provide an accurate determination of trichomes maturation with respect to similar cultivars. At block 450, a time-to-harvest module can be executed to determine an optimum harvest time for the Cannabis biomass. For example, the time-to-harvest module can include inputting a current days of growth relating to the optical image provided by the grower. As described above, the analyzer module can be used to match the image, or series of images if multiple are provided, of the current growth cycle and compare the optical image to all of the historical optical images using any of a number of correlation methods.

In at least one example, a grower may provide 35 optical images of their crop, an optical image for each day from day 1 (germination) through day 35 (current). The series of images can then be matched with corresponding images from the optical history database. The process can be repeated until each of the optical images in the series of images are compared and matched. In at least one example, correlation algorithms can be used in the analysis. Once each of the optical images are matched, the data corresponding to the historical optical images can be used to calculate the number of days left until the crops reach the ideal harvest stage. The time-to-harvest module can be configured to elect a “best match” based on the highest volume and quality of cannabinoids, even if the historical optical images having the highest volume and quality are not the actual best match to the current optical images. The reason for electing a best match relating to volume and quality is due to the fact that there is at least some error associated with the optical images provided by the growers. A recommend harvest date can be determined by correlating the harvest information from the best match images to the growth cycle of the current crop. For example, if the crops indicated in the best match historical images were harvested 15 days after the latest image provided by in the current crop's growth cycle, then a recommended harvest date of 15 days from the last optical image can be estimated.

Finally, at block 460, a recommended harvest date is provided to a grower via the research network. The recommended harvest date, as described above, is estimated on the assumption that the goal of the grower is to achieve the highest volume and quality of cannabinoids possible. However, it should be noted that some growers may base their harvest date on something other than volume and quality of the cannabinoids. As stated above, some growers may determine an earlier harvest day can provide a less tinted, and thus more desirable, extract.

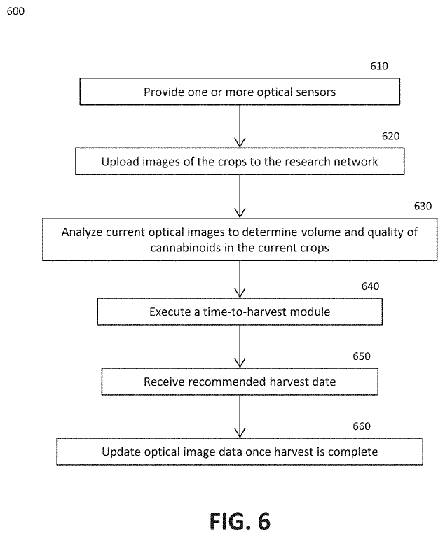

A method 600 for using the grower base module is explained with reference to FIG. 6. The grower's base module can be accessed via a user interface. The user interface can include features such as a communications connector to the extractor, a portal for growers to research on the various databases, and a recommendations interface to receive recommendations from the harvest module. The method 600 for using the grower base module can begin at block 610 by providing one or more optical sensors. As described above, the optical sensors can be hyperspectral cameras or multispectral cameras. These hyperspectral and multispectral cameras can be tuned to detect specific cannabinoids which the growers would like to monitor. At block 620, a plurality of images of the grower's crop, taken at various times throughout the growth cycle of the current crop, are uploaded to the research network.

At block 630, the grower can request an analysis of the optical images via the analyzer module of the research network to extract volume and quality metrics, as described above. The analyzer module can determine the volume and quality data by comparing the current optical images with images from the historical optical database as described above. Since the research network is accessible to various growers, the historical optical database can include images from numerous growers providing a wide variety of different images for comparison. At block 640, the grower can execute the time-to-harvest module as described with respect to FIG. 4. Once the evaluation is complete, at block 650, a recommended harvest date can be provided. As described above, the recommended harvest date is determined based on the assumption that the grower's desired product is one having the highest volume and quality of cannabinoids. For example, the “optimum” volume-quality of the cannabinoids can be different based on the intended Cannabis derivative product. In at least one example, the grower base module can include a description of how to adjust the recommended harvest date based on the grower's desired product if they are not attempting to gain the highest volume or quality cannabinoids.

Finally, at block 660, a grower can access the optical image files uploaded of their crop to update the information. For example, the grower can store the actual harvest date of the crop once the harvest has occurred. Additionally, the grower can input the calculated volume and quality of the cannabinoids extracted from their crop, once the extraction process has been completed. This data can then be stored along with the optical images in the volume-quality database for future reference and comparison. The more updates provided by the growers, the more accurate the time-to-harvest module can be in providing recommended harvest dates.

As stated above, the grower base module allows users to access to all databases stored in the research network. This allows for growers to search the databases for similar plants to view the analyses. For example, if a grower has planted a specific cultivar of Cannabis, they may wish to review the crop analysis provided for others growing the same cultivar to determine the optimum growth period, amount of water, sunlight, nutrients, etc., that may allow the plants to achieve an ideal cannabinoid volume or quality. In at least one example, the research network can include an example optical image showing data relating to intensity vs. wavelength/frequency at any stage during the plant growth cycle. Additionally, the example image can provide a high-quality image indicative of a high volume-quality Cannabis plant from a previous lot, or group of lots.

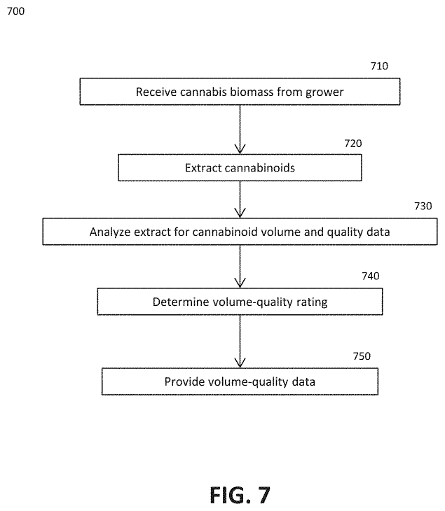

A method 700 for using the extractor base module, described in FIG. 1, is illustrated in FIG. 7. At block 710, the extractors can receive a Cannabis biomass from one or more growers. The extractors can notate the lot number for the Cannabis biomass such that any data relating to the final extraction quality or volume can be properly correlated. At block 720, the extractors can extract the cannabinoids from the Cannabis biomass to obtain a final extracted product. As described above, several methods for the extraction of cannabinoids are known in the art.

At block 730, the extractors can analyze the extracted product using volume and quality sensors, as described with respect to FIG. 1, to determine the volume and quality of the cannabinoids extracted from the Cannabis biomass. In at least one example the volume sensors can include, but are not limited to, analytical lab equipment capable of determining the quantity or concentration of at least THC, CBD, CBN, flavonoids, and terpenes. In at least one example, the quality sensors can include, but are not limited to, analytical lab equipment capable of determining the volume or yield per unit standard of at least THC, CBD, CBN, flavonoids, and terpenes. Once obtained, the volume and quality data can be correlated to the lot number of the cannabinoids, as described above. At block 740, the quality, or concentration, and volume, or yield, rating is determined based on the analytical data. In at least one example, the volume-quality rating can be standardized (high, medium, low), as described above.

Once the volume-quality rating is determined, at block 750 volume-quality data can be provided to the grower and the research network. Providing the final volume and quality information related to the original Cannabis biomass can increase the accuracy of future analyses and harvest date recommendations.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for determining a recommended harvest date, the method comprising:
receiving an optical image of at least one Cannabis plant, the optical image captured by an optical sensor;
retrieving a plurality of historical images from a historical database of images, wherein each of the retrieved historical images is associated with extract data;
comparing the optical image with the plurality of historical images to determine a best matching image; and
making a prediction about an extract of the at least one Cannabis plant based on the extract data associated with the determined best matching image.

2. The method of claim 1, further comprising generating a recommended harvest date based on the prediction about the extract; and sending the generated recommendation to a grower device associated with the received optical image.

3. The method of claim 1, wherein the optical sensor includes at least one of a hyperspectral camera and a multispectral camera, and further comprising tuning the optical sensor to one or more wavelengths associated with one or more compounds targeted for extraction.

4. The method of claim 1, further comprising receiving the plurality of historical images from one or more growers over a period of time, each historical image associated with a different stage in a growth cycle of the at least one Cannabis plant; and storing the plurality of historical images in the historical database.

5. The method of claim 4, wherein receiving the plurality of historical images further includes receiving conditions data for each historical image, the conditions data including at least one of a cultivar, a germination date, a crop location, a light level, a nutrient level, or a water level.

6. The method of claim 1, wherein receiving the plurality of historical images further includes receiving a rating regarding at least one of volume or quality of one or more compounds targeted for extraction.

7. The method of claim 6, wherein the one or more compounds include at least one of delta-9-tetrahydrocannabinol (THC), cannabidiol (CBD), cannabinol (CBN), and combinations thereof.

8. The method of claim 1, wherein making the prediction includes estimating a current rating of at least one of volume or quality of the at least one Cannabis plant in the captured optical image.

9. The method of claim 1, wherein comparing the optical image with the plurality of historical images includes generating a graph of one or more intensities within the captured optical image over one or more wavelengths associated with the captured optical image; and comparing the graph to one or more corresponding graphs associated with each of the historical images.

10. A system for determining a recommended harvest date, the system comprising:
a communication network interface that:
receives an optical image of at least one Cannabis plant, the optical image captured by an optical sensor, and
retrieves a plurality of historical images from a historical database of images, wherein each of the retrieved historical images is associated with extract data; and
a processor that executes instructions stored in memory, wherein the processor executes the instructions to;
compare the optical image with the plurality of historical images to determine a best matching image; and
make a prediction about an extract of the at least one Cannabis plant based on the extract data associated with the determined best matching image.

11. The system of claim 10, wherein the processor further generates a recommended harvest date based on the prediction about the extract; and wherein the communication network interface sends the generated recommendation to a grower device associated with the received optical image.

12. The system of claim 10, wherein the optical sensor includes at least one of a hyperspectral camera and a multispectral camera, and wherein the optical sensor is tuned to one or more wavelengths associated with one or more compounds targeted for extraction.

13. The system of claim 10, wherein the communication network interface further receives the plurality of historical images from one or more growers over a period of time, each historical image associated with a different stage in a growth cycle of the at least one Cannabis plant; and further comprising memory of the historical database that stores the plurality of historical images.

14. The system of claim 13, wherein the communication network interface further receives conditions data for each historical image, the conditions data including at least one of a cultivar, a germination date, a crop location, a light level, a nutrient level, or a water level.

15. The system of claim 10, wherein the communication network interface further receives a rating regarding at least one of volume or quality of one or more compounds targeted for extraction.

16. The system of claim 15, wherein the one or more compounds include at least one of delta-9-tetrahydrocannabinol (THC), cannabidiol (CBD), cannabinol (CBN), and combinations thereof.

17. The system of claim 10, wherein the processor makes the prediction by estimating a current rating of at least one of volume or quality of the at least one Cannabis plant in the captured optical image.

18. The system of claim 10, wherein the processor compares the optical image with the plurality of historical images by generating a graph of one or more intensities within the captured optical image over one or more wavelengths associated with the captured optical image; and comparing the graph to one or more corresponding graphs associated with each of the historical images.

19. A non-transitory, computer-readable storage medium, having embodied thereon a program executable by a processor to perform a method for determining a recommended harvest date, the method comprising:
receiving an optical image of at least one Cannabis plant, the optical image captured by an optical sensor;
retrieving a plurality of historical images from a historical database of images, wherein each of the retrieved historical images is associated with extract data;
comparing the optical image with the plurality of historical images to determine a best matching image; and
making a prediction about an extract of the at least one Cannabis plant based on the extract data associated with the determined best matching image.
